(19) United States
(12) Patent Application Publication
(10) Pub. No.: US 2007/0038243 A1
(43) Pub. Date: Feb. 15, 2007

(54) Tactical combat tourniquet

(76) Inventor: David B. Rutherford, Boca Raton, FL (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 11/410,638

(22) Filed: Apr. 25, 2006

Related U.S. Application Data
(60) Provisional application No. 60/675,032, filed on Apr. 26, 2005.

(51) Int. Cl. A61B 17/00

(52) U.S. Cl. 606/203

(57) ABSTRACT

A tourniquet is provided which is specifically structured to be easily and quickly applied to an extremity of a body and to be securely positioned in place on the extremity, the tourniquet being particularly structured for self-use by a wounded person under the stressful conditions of the battlefield and being structured to be used single-handedly.

### CROSS-REFERENCE TO RELATED APPLICATION

This application is a non-provisional application claiming priority to provisional patent application Ser. No. 60/675,032 filed Apr. 26, 2005.

### BACKGROUND OF THE INVENTION

1. Field of the Invention

This invention relates to a tourniquet for restricting blood flow to the extremities of the body in the emergency pre-hospital environment of tactical combat. In particular, the present invention relates to a tourniquet that is structured for improved application in battlefield conditions to restrict blood flow to effected extremities and to remain in place in a secure fashion under the stressful conditions of combat.

2. Description of Related Art

Historically, hemorrhage has been the leading cause of death on the battlefield. Thus, tourniquets have been in use since the seventeenth century in battlefield conditions to manage blood loss resulting from severe wounds. The use of a tourniquet to manage life-threatening hemorrhaging in extremities has been essential.

A tourniquet is basically a compression device which is placed about a bodily extremity, such as an arm or a leg, to cut off the flow of blood to a part of the body. A tourniquet can be made from a variety of materials and/or objects, but most typically comprises a ligature of fabric or leather that encircles a limb. The ligature is structured for tightening about the limb to provide compression of the vascular system in that limb.

While the many types of tourniquets that have been developed are each useful for the intended purpose, they have certain limitations of use which do not render them suitable for use under battlefield conditions. Known tourniquet devices, for example, tend to be structurally complicated making it necessary for only skilled medical workers to apply them. Also, some known tourniquet devices have complicated apparatus for providing compression, such as inflatable portions, and such devices become very difficult to apply and inflate to provide the necessary compression.

It is particularly desirable in battlefield conditions for a tourniquet to be easily and quickly applied to a wounded extremity. Moreover, it would be desirable for a tourniquet to be usable by the injured person to prevent exsanguination before more extensive or professional medical care can be administered. It would also be important for such a tourniquet device to be structured to stay securely in place while the casualty victim is moved to cover for further treatment. Prior art devices tend to be difficult to handle or use, and are typically not manipulable single-handedly by the victim to address his own hemorrhaging condition under the stress of the battlefield.

### BRIEF SUMMARY OF THE INVENTION

In accordance with the present invention, a tourniquet is provided which is specifically structured to be easily and quickly applied to an extremity of a body and to be securely positioned in place on the extremity. The tourniquet of the present invention is particularly structured for self-use by a wounded person, and is structured to be used single-handedly.

The tourniquet of the present invention generally comprises a band sized in length to encircle an extremity of the body (e.g., an arm or leg). The band is further structured with apparatus for securely positioning the band about the extremity. The apparatus for securing the band is structured to be manipulable with a single hand to facilitate application of the invention by a wounded person to himself in battlefield conditions.

The tourniquet of the present invention further includes apparatus for providing further compression of the tourniquet against the extremity to selectively increase the amount of pressure exerted on the vascular system in the extremity. The apparatus for selectively increasing compression is also structured to be usable and manipulable single-handedly, and is structured with a securement device for securing the apparatus in place once a desired amount of additional compression is achieved.

The tourniquet of the present invention may be structured for use on both humans and animals, but is described herein with respect to its use on humans in battlefield conditions.

### BRIEF DESCRIPTION OF THE SEVERAL VIEWS OF THE DRAWINGS

In the drawings, which currently illustrate the best mode for carrying out the invention:

FIG. 1 is a plan view of the tourniquet of the present invention;

FIG. 2 is a perspective view of an apparatus for selectively providing additional compression;

FIG. 3 depicts the upper arm of a person and illustrates the placement of the tourniquet of the present invention on an arm; and

FIG. 4 depicts the leg of a person and illustrates the placement of the tourniquet of the present invention on a leg.

### DETAILED DESCRIPTION OF THE INVENTION

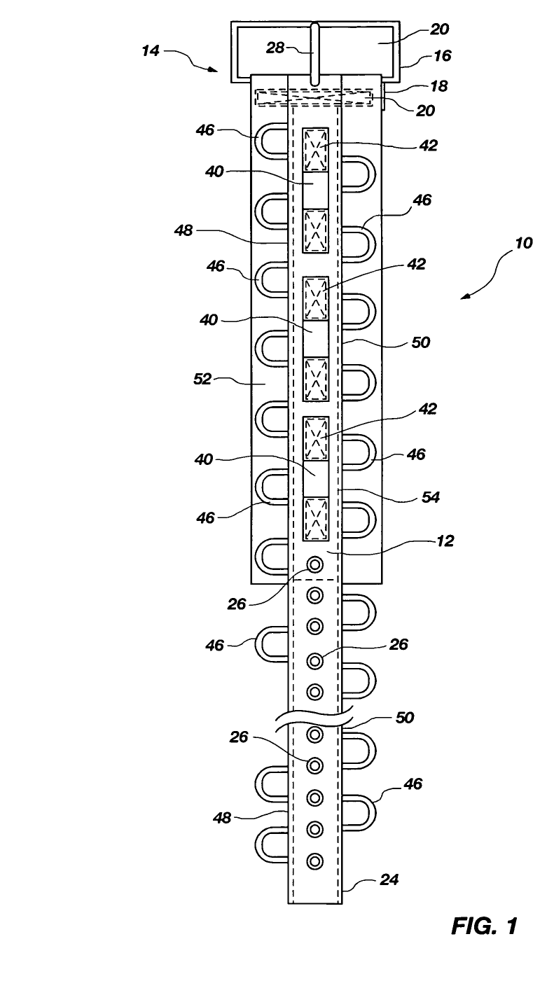

FIG. 1 illustrates the tourniquet 10 of the present invention which generally comprises a band 12 of material sized in length to encircle an extremity of a body, such as an arm or a leg. The band 12 is made of any suitable material that provides sufficient flexibility to apply the band 12 about an extremity and to provide sufficient compression to the extremity when positioned in place. The band 12, therefore, may optimally be made of a material that has very little or no longitudinal elasticity, although some longitudinal elasticity may be suitable.

The tourniquet 10 of the invention is also provided with apparatus 14 for securing the tourniquet 10 in position about an extremity. The apparatus 14 may be any suitable device or element, but is illustrated in FIG. 1, by way of example only, as a conventional buckle 16 that is secured to a first end 18 of the band 12 by appropriate means, such as stitching 20. Other forms or means of attaching the buckle 16 to the first end 18 of the band 12 may be equally suitable.

The opening 22 of the buckle 16 is sized to receive the second end 24 of the band 12 therethrough, and a plurality of eyelets 26 are formed along the length of the band 12 for positioning of the buckle tongue 28 through the eyelets. The plurality of eyelets 26 enables the tourniquet 10 to be applied and secured to extremities of various circumference.

The tourniquet 10 of the present invention is further provided with apparatus for selectively increasing the degree of compression exerted on the extremity by the tourniquet 10 after the band 12 has been appropriately applied to the extremity. That is, once the band 12 has been applied to an extremity and tightened to a desired degree of compression, it may be desirable or necessary to apply a further incremental increase in compression to the extremity to stop blood flow through the vascular system in the extremity. Accordingly, the apparatus 30 for selectively increasing the degree of compression is configured to provide an incremental amount of increased compression that is not achievable by simply tightening the band 12 through movement of the tongue 28 of the buckle 16 to the next eyelet 26.

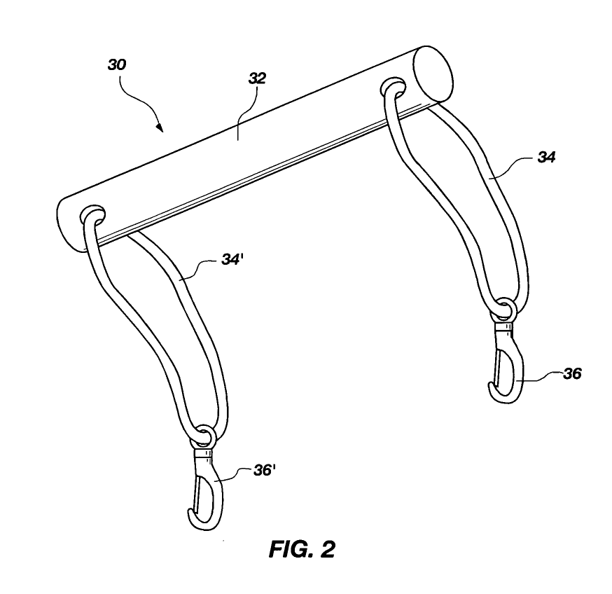

One exemplar apparatus 30 for selectively increasing the degree of compression is shown in FIG. 2 and comprises a rod or dowel 32 having at each end thereof a cord 34, 34′ attached to the dowel 32. A securement device, such as a hook 36, 36′ is attached to each of the cords 34, 34′ and provides means for securing the dowel 32 in place once a desired amount of incremental increase in compression is achieved.

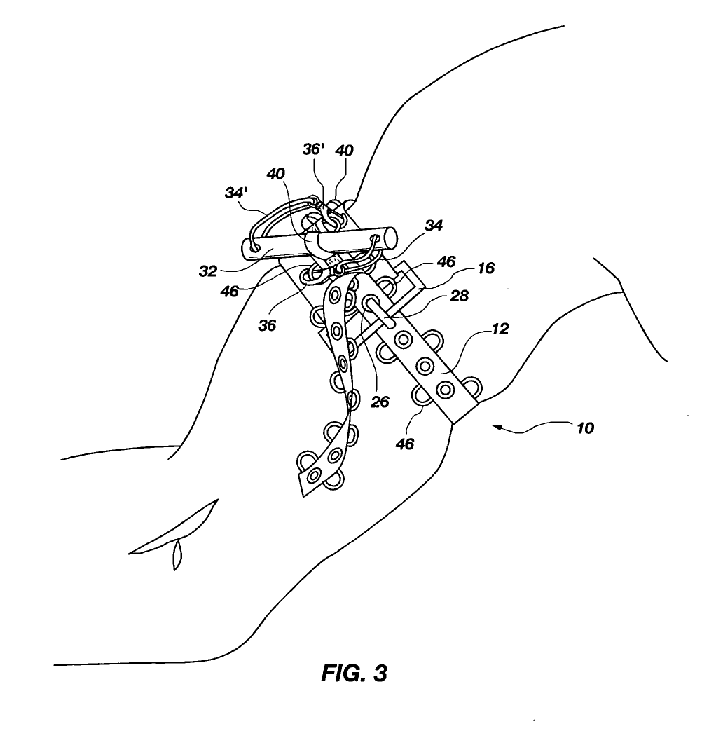

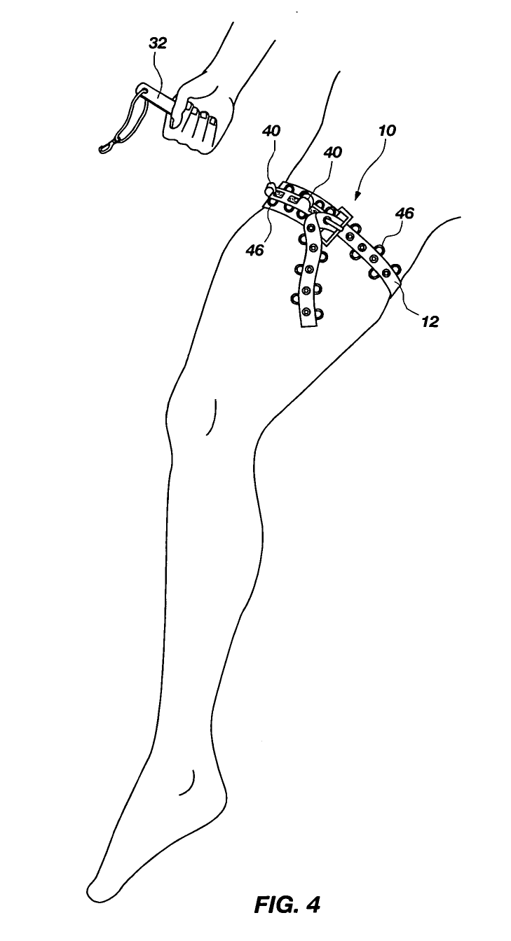

Referring again to FIG. 1, the tourniquet 10 is structured to accommodate the dowel 32 for providing incremental compression to the extremity. That is, the band 12 is structured with at least one loop 40 (here shown having a plurality of loops 40) which extends upwardly from the band 12, as best seen in FIGS. 3 and 4. The loop 40 may, for example, be formed by securement of a strip of material 42 to the band 12 by such means as stitching 44.

As best seen in FIG. 3, where the tourniquet 10 is shown attached to an upper arm, the loops 40 of the tourniquet 10 are sized to receive the dowel 32 therethrough. Once the dowel 32 is positioned in an upwardly extending loop 40, the dowel 32 is then twisted to apply increased tension on the band 12 and thereby incrementally increase the amount of compression applied to the extremity. Once a selected amount of increased compression is achieved, the position of the dowel 32 in the loop 40 is maintained by securing the hooks 36, 36′ associated with the dowel 32 to one of a plurality of side loops 46 that are positioned along the outer margins 48, 50 of the band 12, as further shown in FIG. 1. The hooks 36, 36′, each attached to one of the side loops 46, will be positioned generally at 180° from each other to thereby keep the dowel 32 in its selected twisted position.

The tourniquet 10 of the present invention may further include a second band or strap 52 to which the band 12 is attached by any suitable means, such as stitching 54. The strap 52 may, most suitably, be wider than the band 12 and, as such, provides increased surface area for effecting compression of the extremity. The strap 52 may be sized in length to encircle all or a portion of the extremity. If the tourniquet 10 is structured with a strap 52, it is preferred that the tourniquet 10 be positioned on the extremity so that the strap 52 is positioned over the vascular structure that requires compression to stop the bleeding (i.e., over the major artery if the wound is arterial or over the major vein if the wound is venous) so that the optimal amount of compression can be achieved.

It may be seen that the tourniquet 10 of the present invention is simply constructed to facilitate quick and easy application of the tourniquet to a wounded extremity. It is also seen that the tourniquet of the present invention is easily manipulable with one hand and can be applied by the user to his own body in the battlefield. The compact construction enables the tourniquet to be easily stored about a soldier's person or personal effects in the battlefield, thereby increasing a soldier's ability to provide immediate care to himself or to others until professional or more extensive medical aid can be provided.

The tourniquet of the present invention can be constructed in any number of ways to provide an easily used device with selectively adjustable incremental compression. Hence, reference herein to specific structures or functions of the invention are by way of example, and not by way of limitation.

What is claimed is:

1. A tourniquet, comprising:
a band sized in length to encircle an extremity of a body;
apparatus for securing said band in position about an extremity, said apparatus for securing said band being structured to be manipulable with a single hand to facilitate application of said band by a wounded person to himself in battlefield conditions; and
apparatus for providing further compression of said band against the extremity to selectively increase the amount of pressure exerted on the vascular system in the extremity.

2. The tourniquet of claim 1 wherein said apparatus for securing said band in position about an extremity further comprises a buckle, and wherein said band is structured with a plurality of eyelets formed along said length of said band for receiving a tongue of said buckle.

3. The tourniquet of claim 1 wherein said apparatus for providing further compression of said band against the extremity further comprises a dowel attachable to said band for incrementally increasing compression of said band against an extremity, said dowel further being structured with a securement device for securing the dowel in position on said band once an incremental increase in compression is achieved.

4. The tourniquet of claim 3 wherein said band further comprises at least one loop which extends upwardly from said band and is sized to receive said dowel therethrough.

5. The tourniquet of claim 4 wherein said band is structured with side margins and further comprises a plurality of side loops extending from said side margins of said band to provide apparatus for securing said securement device to said band.

6. The tourniquet of claim 5 further comprising a strap sized to be wider in dimension than said band and being secured to said band along at least a portion of said length of said band.

7. The tourniquet of claim 6 wherein said apparatus for securing said band in position about an extremity further comprises a buckle, and wherein said band is structured with a plurality of eyelets formed along said length of said band for receiving a tongue of said buckle.

8. The tourniquet of claim 1 further comprising a strap sized to be wider in dimension than said band and being secured to said band along at least a portion of said length of said band.

9. A tourniquet, comprising:
a band sized in length to encircle an extremity of a body, said band having a first end and a second end and side margins;
a buckle attached to said first end of said band;
a plurality of eyelets formed along said length of said band for securement to said buckle;
at least one loop extending upwardly from said band;
a plurality of side loops extending from said side margins of said band; and
a dowel sized to be received in said at least one loop, said dowel having at least one hook for attachment to one of said side loops for securement of said dowel in said at least one loop.

10. The tourniquet of claim 9 further comprising a strap attached to said band and being sized in width dimension to be wider than said band.

11. The tourniquet of claim 9 wherein said dowel is configured with two hooks for attachment of each said hook to one of said side loops for securement of said dowel in position relative to said band.
